(19) United States
(12) Patent Application Publication
(10) Pub. No.: US 2021/0363127 A1
(43) Pub. Date: Nov. 25, 2021

(54) PROCESS FOR THE PREPARATION OF 2,2-DIFLUORO-1,3-BENZODIOXOLE AND INTERMEDIATES THEREOF

(71) Applicant: SRF LIMITED, Gurgaon (IN)

(72) Inventors: Radha Kumarasamy, Gurgaon (IN); Poornachandran Ravichandran, Gurgaon (IN); Aiyswariya Rajaram, Gurgaon (IN); Prasannakumar Seetharaman, Gurgaon (IN); Arumugam Nagappan, Gurgaon (IN); Kapil Kumar, Gurgaon (IN); Anurag Jain, Gurgaon (IN)

(21) Appl. No.: 17/282,268

(22) PCT Filed: Oct. 4, 2019
(86) PCT No.: PCT/IN2019/050735

§ 371 Date: Apr. 1, 2021

(30) Foreign Application Priority Data
Oct. 4, 2018 (IN) 201811037656

(51) Int. Cl. C07D 317/68

(52) U.S. Cl. CPC C07D 317/68

(57) ABSTRACT

A process for preparing 2,2-difluoro-1,3-benzodioxole is provided that comprises a step of reacting 1,3-benzodioxole with chlorine in benzotrifluoride in the presence of a radical initiator. Additional processes are provided for preparing 2,2-dichloro-1,3-benzodioxole, which is used as an intermediate in preparation of 2,2-difluoro-1,3-benzodioxole.

### CROSS-REFERENCES TO RELATED APPLICATIONS

This application is a national-stage application under 35 U.S.C. § 371 of International Application PCT/IN2019/050735, filed Oct. 4, 2019, which claims the benefit of priority to Indian Patent Application No. 201811037656, filed Oct. 4, 2018.

### TECHNICAL FIELD

This application relates to a process for preparing 2,2-difluoro-1,3-benzodioxole. The process comprises a step of reacting 1,3-benzodioxole with chlorine in benzotrifluoride in the present of a radical initiator. This application also relates to the process for preparing 2,2-dichloro-1,3-benzodioxole which is used as an intermediate in preparation of 2,2-difluoro-1,3-benzodioxole.

### BACKGROUND

2,2-difluoro-1,3-benzodioxole, is used as an important intermediate for synthesizing the agrochemical and pharmaceutical products.

The EP Patent No. 1,502,908 provides a process for the preparation of 2,2-dichloro-1,3-benzodioxole by reacting 1,3-benzodioxole with chlorine in the presence of a radical initiator, in dichlorobenzotrifluoride as solvent. The 2,4-dichlorobenzotrifluoride used in the process as solvent has boiling point of 117° C., which is very close to the boiling point of 2,2-difluoro-1,3-benzodioxole (130° C.). Thus, there arises a difficulty in the separation of product from solvent.

The inventors have found a process for the preparation of fluorinated derivatives of ethers that overcome the drawbacks of the existing processes.

### SUMMARY

The main object of the present disclosure is to provide an industrially advantageous, efficient, and safe process for the preparation of 2,2-difluoro-1,3-benzodioxole.

The first aspect relates to a process for preparation of 2,2-difluoro-1,3-benzodioxole, comprising the steps of:

(a) adding an aqueous solution of a base to catechol to obtain a reaction mixture 1;

(b) adding reaction mixture 1 to a mixture of phase transfer catalyst and dichloromethane;

(c) heating the step (b) result to obtain 1,3-benzodioxole;

(d) reacting 1,3-benzodioxole with chlorine in presence of benzotrifluoride and a radical initiator to obtain 2,2-dichloro-1,3-benzodioxole

(e) venting out the hydrochloric acid formed;

(f) reacting hydrogen fluoride with 2,2-dichloro-1,3-benzodioxole in presence of benzotrifluoride;

(g) venting out excess of hydrogen fluoride; and

(h) isolating 2,2-difluoro-1,3-benzodioxole.

The second aspect relates to a process for preparation of 2,2-difluoro-1,3-benzodioxole, comprising the steps of:

(a) adding an aqueous solution of a base to catechol to obtain a reaction mixture 1;

(b) adding reaction mixture 1 to a mixture of phase transfer catalyst and dichloromethane;

(c) heating the step (b) result to obtain 1,3-benzodioxole;

(d) reacting 1,3-benzodioxole with chlorine in presence of benzotrifluoride and a radical initiator to obtain 2,2-dichloro-1,3-benzodioxole

(e) venting out the hydrochloric acid formed; and

(f) converting the compound of 2,2-dichloro-1,3-benzodioxole to 2,2-difluoro-1,3-benzodioxole.

The third aspect relates to a process for preparation of 2,2-difluoro-1,3-benzodioxole, comprising the steps of:

(a) reacting 1,3-benzodioxole with chlorine in presence of benzotrifluoride and a radical initiator to obtain 2,2-dichloro-1,3-benzodioxole

(b) venting out the hydrochloric acid formed;

(c) reacting hydrogen fluoride with 2,2-dichloro-1,3-benzodioxole in presence of benzotrifluoride;

(d) venting out excess of hydrogen fluoride; and

(e) isolating 2,2-difluoro-1,3-benzodioxole.

### DETAILED DESCRIPTION

As used herein, the term “base” refers to sodium hydroxide, potassium hydroxide, sodium hydride, potassium hydride, sodium carbonate, sodium bicarbonate, potassium carbonate, potassium bicarbonate or the like.

As used herein, fluorination refers to reacting 2,2-dichloro-1,3-benzodioxole with hydrogen fluoride.

As used herein, chlorination refers to reacting 1,3-benzodioxole with chlorine.

As used herein, the term “phase transfer catalyst” refers to quaternary salts of ammonium, phosphonium or arsonium chlorides and bromides. The examples of suitable phase transfer catalyst include tetrabutylammonium bromide (TBAB), methyltriethylammonium chloride (MTAC), triethylbenzylammonium chloride (TBAC), tetrabutylphosphonium bromide (TBPB) or the like.

As used herein, a radical initiator refers UV light, benzoyl peroxide, diacetyl peroxide, succinyl or azobisisobutyronitrile (AIBN) are suitable. Radical initiators are used in amounts of from 0 to 10 wt %.

Dichloromethane may be used as its mixture with a solvent selected from a group consisting of toluene, sulfolane, chlorobenzene, nitrobenzene, acetonitrile, benzotrifluoride or the like.

As used herein, the term “isolating” refers to the method used to isolate the compound from the reaction mixture. The isolation is carried out using any of the process consisting of extraction, distillation, filtration, decantation, washing, dryings or combination thereof

As used herein, the term “venting out” refers to the method of removing the gaseous compound either by heating, purging an inert gas or both.

As used herein, the term “inert gas” refers to nitrogen, helium or argon.

An embodiment of the first aspect provides a process for preparation of 2,2-difluoro-1,3-benzodioxole, wherein the step (b) is carried out at a temperature of 100° C. to a temperature of 120° C. at a pressure of 7 to 8 kg/cm².

In another embodiment, the step of “reacting 1,3-benzodioxole with chlorine” is performed by purging chlorine gas into a mixture of 1,3-benzodioxole, AIBN catalyst and benzotrifluoride at a temperature of 80° C. to a temperature of 100° C.

In another embodiment, 2,2-dichloro-1,3-benzodioxole is not isolated and its reaction mixture with benzotrifluoride is carried forward to the next step.

In another embodiment, the step of “reacting hydrogen fluoride with 2,2-dichloro-1,3-benzodioxole” is performed by adding hydrogen fluoride to a mixture of a 2,2-dichloro-1,3-benzodioxole and benzotrifluoride at a temperature of 0° C. to a temperature of 10° C.

In another embodiment, the unreacted hydrogen fluoride is vented out either by heating or by purging an inert gas and additionally by washing the reaction mixture with an aqueous solution of sodium bicarbonate.

### EXAMPLES

### Example 1

### Preparation of 1,3-Benzodioxole

Tetrabutyl ammonium bromide (0.026 moles) in dichloromethane (5 moles) was added in the pressure reactor vessel at room temperature and heated to 105° C.-110° C. The pressure raised up to 7-8 kg/cm². A mixture of catechol (1 mole) and sodium hydroxide (2.0 moles of sodium hydroxide dissolved in 20.0 moles of water), was dosed through pump to the pressure reactor for 3 hours. The pressure was raised to 7-8 kg/cm². The reaction mass was heated and maintained for 1.0 hours at 110° C. The pressure was gradually decreased from 8 to 4 kg/cm². The reaction mass was cooled down to 25-30° C. The resultant mass was filtered through Buckner funnel using Celite bed. The filtered mass was taken for layer separation. The product was washed using dichloromethane (5.0 moles). The combined crude organic layer was taken for distillation. The crude organic layer was distilled at atmospheric condition to remove dichloromethane followed by distillation at reduced pressure to obtain the titled product. Yield: 80%. Purity 95%.

### Example 2

### Comparative Example

### Preparation of 1,3-Benzodioxole

Dichloromethane (1.56 g., 100 mL) and TBAB (0.02 moles) are placed in an autoclave, and to this mixture is added catechol (0.2 moles) and caustic soda (0.6 moles) in flake form, with agitation, at 80° C. After the reaction is completed, the titled compound is recovered by following the procedure described in Example 1, that is, the organic phase is separated, and excess methylene chloride is distilled off and recycled. Yield 60%. Purity: 65%.

### Example 3

### Preparation of 2,2-dichloro-1,3-benzodioxole

Chlorine (2.05 moles) was added to a mixture of 1,3-benzodioxole (1.0 mole) in benzotrifluoride (2.0 moles) and AIBN (0.05%) at 85-95° C. for 3 hours and the mass was stirred at the same temperature for one hour for reaction completion. Then the reaction mass was cooled to room temperature and the nitrogen gas was passed to remove unreacted dissolved chlorine and hydrogen chloride. The crude 2,2-dichloro-1,3-benzodioxole in benzotrifluoride is taken as such to next step. Yield 88%. Purity 97%.

### Example 4

### Preparation of 2,2-dichloro-1,3-benzodioxole

Chlorine (2.05 moles) was added to a mixture of 1,3-benzodioxole (1.0 mole) in benzotrifluoride (2.0 moles) and benzoyl peroxide (0.05 moles) at 80-90° C. for 2 hours and the mass was stirred at the same temperature for one hour for reaction completion. Then the reaction mass was cooled to room temperature and the nitrogen gas was passed to remove unreacted dissolved chlorine and hydrogen chloride. The crude 2,2-dichloro-1,3-benzodioxole in benzotrifluoride is taken as such to next step. Yield 88%. Purity 97%.

### Comparative Example

### Preparation of 2,2-dichloro-1,3-benzodioxole

In a stirred apparatus with a chlorine inlet, metering, intensive condenser and gas outlet, solution of AIBN (0.05%) in 1,3-benzodioxole (1.0 mole) and chlorine (2.05 moles) are simultaneously introduced in the reactor. After termination of the dosage was blown at 130° C. with nitrogen. The titled compound was distilled out. Yield: 66%. Purity: 74%.

### Example 5

### Preparation of 2,2-difluoro-1,3-benzodioxole

2,2-dichloro-1,3-benzodioxole (2.0 moles) was continuously added to hydrogen fluoride (4.0 moles) in an autoclave at a temperature of 0° C. for 2-3 hours. After completion of the addition, the reaction mass was stirred at 0° C. for an hour to achieve complete conversion. The excess hydrogen fluoride was vented off by heating the mass up to 80° and the adhered hydrogen fluoride was removed by washing with sodium by carbonate solution. Then the reaction mass was distilled through two meter distillation column under vacuum to get the product. Yield: 86%. Purity: 94%.

1-10. (canceled)

11. A process for preparation of 2,2-difluoro-1,3-benzodioxole, the process consisting of:
(a) adding an aqueous solution of a base to catechol to obtain a first reaction mixture;
(b) adding the first reaction mixture to a mixture of phase transfer catalyst and dichloromethane at a pressure of 7 kg/cm² to 8 kg/cm² to obtain a second reaction mixture;
(c) heating the second reaction mixture at a temperature of 100° C. to 120° C. to obtain 1,3-benzodioxole;
(d) reacting the 1,3-benzodioxole with chlorine in the presence of benzotrifluoride and a radical initiator at a temperature of 80° C. to 100° C. to obtain 2,2-dichloro-1,3-benzodioxole;
(e) venting out the hydrochloric acid formed in (d);
reacting hydrogen fluoride with the 2,2-dichloro-1,3-benzodioxole in the presence of benzotrifluoride;
(g) venting out excess hydrogen fluoride from (f); and
(h) isolating 2,2-difluoro-1,3-benzodioxole, wherein (d), (e), and (f) are carried out without isolation of 2,2-dichloro-1,3-benzodioxole.

12. The process of claim 1, wherein the base in (a) is selected from the group consisting of sodium hydroxide, potassium hydroxide, sodium hydride, potassium hydride, sodium carbonate, sodium bicarbonate, potassium carbonate, potassium bicarbonate, and mixtures thereof.

13. The process of claim 1, wherein the phase transfer catalyst of (b) is selected from quaternary salts of ammonium chlorides and quaternary salts of ammonium bromides.

14. The process of claim 1, wherein reacting hydrogen fluoride with the 2,2-dichloro-1,3-benzodioxole in (f) is carried out at a temperature of 0° C. to 10° C.

15. The process of claim 1, wherein the radical initiator is selected from the group consisting of UV light, benzoyl peroxide, diacetyl peroxide, succinyl, and azobisisobutyronitrile.
